(19) United States
(12) Patent Application Publication
(10) Pub. No.: US 2016/0213027 A1
(43) Pub. Date: Jul. 28, 2016

(54) PET FOOD ADDITIVE

(71) Applicant: GEORGE MANIATAKOS, FT LAUDERDALE, FL (US)

(72) Inventor: GEORGE MANIATAKOS, FT LAUDERDALE, FL (US)

(21) Appl. No.: 15/002,683

(22) Filed: Jan. 21, 2016

Related U.S. Application Data
(63) Continuation of application No. 62/125,440, filed on Jan. 21, 2015.

(51) Int. Cl. A23L 1/226; A61K 31/05; A61K 35/60; A61K 8/22; A61K 35/00; A61K 31/122; A61K 8/20; A61K 36/53; A61K 31/375; A23K 1/00

(52) U.S. Cl. CPC A23K 1/1846; A61K 36/53; A61K 31/05; A61K 35/60; A61K 8/22; A61K 31/375; A61K 35/00; A61K 31/122; A61K 8/20; A23L 1/231; A61Q 11/00

(57) ABSTRACT

This invention relates to a system and compositions for providing pet food products having improved palatability and nutrition and, in particular to a system for selectively providing varied flavor in a pet food diet.

The present application is a non-provisional US patent application which claims benefit under 35 USC §119(e) of the U.S. Provisional patent Application Ser. No. 62/125,440 filed Jan. 21, 2015.

### FIELD OF THE INVENTION

The invention relates to a system and compositions for providing pet food products having improved palatability and nutrition and, in particular to a system for selectively providing varied flavor in a pet food diet.

### BACKGROUND OF THE INVENTION

Pet foods, particularly cat foods and dog foods, are commercially available in wet, dry, or semi-dry form, in various sizes and types of packaging. Food products designed for dogs, cats, or other household pets, are commonly made from meat, meat by-products, fish, fish by-products, cereals and grains and may also include vitamins and minerals. Typically such products are nutritious, but may not be highly savory or palatable.

Various methods and compositions are known for enhancing the palatability of pet foods by the addition flavorants or palatability enhancing compounds. U.S. Pat. No. 3,203,806 to Young discloses enhancing the palatability of pet foods by the addition of a flavor-enhancing additive containing a small amount of acetamide and acetic acid.

U.S. Pat. No. 4,070,490 to Lugay et al discloses a pet food having the flavor and texture of animal liver prepared by a process that comprises heating a mixture of water, emulsified fat, reducing sugar and blood.

U.S. Pat. No. 4,215,149 to Majlinger discloses a process for improving the palatability of particulate dry pet food by coating the surface of the particulates with fat and then applying a salt of phosphoric acid to the fat-coated surface.

U.S. Pat. No. 4,267,195 to Boudreau et al discloses the addition of certain palatability enhancing compounds to dog foods. The compounds applied include L-proline, L-cysteine, L-histidine, L-lysine, inosine 5′-triphosphate, inosine 5′-diphosphate, and adenosine 5′-triphosphate.

U.S. Pat. No. 4,393,085 to Sporadlin et al discloses a process for the preparation of dog food wherein the palatability of the product is improved by treating a farinaceous component of the food with both protease and amylase enzymes.

U.S. Pat. No. 5,186,964 to Gierhart et al discloses a method for increasing the palatability of dry cat food by the application of a surface coating of a sodium acid pyrophosphate, optionally together with an organic acid and a flavorant.

U.S. Pat. No. 6,379,727 to Addy describes a flavoring system for pets that includes as its base maltodextrin with tricalcium phosphate and oleo-resin flavorings.

Although the prior art discloses various processes and compositions for enhancing the palatability of pet foods, it will be appreciated by those skilled in the art that a need continues to exist for improved methods and compositions for this purpose as well as a convenient and economical method for providing variety in the flavor and taste of pet foods as well as providing additional nutritional or health benefit for the pet.

### SUMMARY OF THE INVENTION

It is an object of the present invention to provide pet foods of improved palatability as well as provide additional nutritional or health benefit.

It is a further object of the present invention to provide a process and composition that can be conveniently applied to a base pet food without the need for elaborate or complex processing equipment, to improve the palatability and flavor thereof.

It is a still further object to provide a system, suitable for home use, whereby a pet owner can conveniently and economically provide a flavor-varied diet for a household pet or provide a flavored supplement to provide additional nutritional or health benefit to pets.

The above and other objects are accomplished in accordance with the present invention which comprises a flavoring system for pet food products based on liquid flavor additive composition which may be topically applied to a dry or moistened pet food to enhance the palatability thereof and add flavor thereto.

The present invention relates to a non-aqueous composition for applying onto pet food in which the composition has one or more pet food flavorants. The compositions of the present invention include non-aqueous compositions which are comprised of more than 80 mass % or more than 90 mass % or more than 95 mass % or more than 97 mass % oil. The present invention relates compositions where the oil in the composition is selected from the group consisting of safflower oil, soybean oil, olive oil, flaxseed oil, cottonseed oil, corn oil, canola oil and sunflower oil and combinations thereof. The present invention relates to pet food additive compositions having cannabidiol oil and/or fish oil and/or rosemary oil and/or hydrogen peroxide. Flavorants include but are not limited to bacon flavor, desiccated liver and combinations thereof. The present invention relates to non-aqueous compositions comprising from about 40 to about 60 mass % safflower oil, from about 30 to about 50 mass % flaxseed oil, from about 0.01 to about 0.05 mass % cannabidiol oil, from about 1.0 to about 5 mass % fish oil, from about 0.01 to about 0.1 mass % rosemary oil, from about 0.1 to about 2 mass % hydrogen peroxide, and from about 0.01 to about 5.0 mass % flavorant.

### DETAILED DESCRIPTION

The flavor additive composition may be dissolved or dispersed in a liquid medium, preferably an non-aqueous medium, and sprayed on a base pet food, for example with a hand sprayer, or added with mixing to a package of moist pet food. In another alternate method, a liquid flavor additive composition may be employed directly without prior dissolution and applied to the base pet food. The typical preparation of this non-aqueous liquid product is a base of oils. Suitable oils include but are not limited to safflower oil, soybean oil, olive oil, cottonseed oil, corn oil, canola oil, flaxseed oil and sunflower oil. The base oils may be greater than about 80% or greater than about 85% or greater than about 90% or greater than about 95% or greater than about 96% or greater than about 97% or greater than about 98% or greater than about 99% of the formulation. The base oils may be about 80% to about 90% or about 80% to about 90% or about 80% to about 95% or about 80% to about 96% or about 80% to about about 97% or about 80% to about 98% or about 80% to about 99% or from about 85% to about 90% or about 85% to about 90% or about 85% to about 95% or about 85% to about 96% or about 85% to about 97% or about 85% to about 98% or about 85% to about 99% or from about 90% to about 95% or about 90% to about 96% or about 90% to about 97% or about 90% to about 98% or about 90% to about 99% or from about 95% to 96% or about 95% to about 97% or about 95% to about 98% or about 95% to about 99% of the formulation. Additional oils added to the base that may have medicinal value include but are not limited to rosemary oil (benefits indigestion, mouth care and skin care), cannabidiol (CBD) oil (antiemetic), fish oil (has EPA and DHA and helps heart health and blood pressure). In some embodiments the additional oil(s) may be between about 0.01% to about 5% or from about 0.01% to about 2.5% or from about 0.01% to about 2.0% or from about 0.01% to about 1.5% or from about 0.01% to about 1% or from about 0.01% to about 0.5% or from about 0.01% to about 0.1% or from about 0.01% to about 0.05% or from about 0.05% to about 5.0% or from about 0.05% to about 2.5% or from about 0.05% to about 2% or from about 0.05% to about 1.0% or from about 0.05% to about 0.5% or from about 0.1% to about 5.0% or from about 0.1% to about 2.5% for from about 0.1% to about 1.5% or from about 0.1% to about 1.0% or from about 0.1% to 0.5% of the formulation.

The base oil(s) may be placed into a mixing vat and receives the flavorings and a preservative/antioxidant such as vitamin E or lecithin. The solution is mixed until uniform and homogeneous. This liquid product has the advantage of being placed onto the food substrate more evenly than powder and allows addition directly to the food substrate without premixing. In some embodiments the total amount of flavorant (there may be more than one flavorant in a given composition) may be present in a trace amount or may be between about 0.01% to about 5% or from about 0.01% to about 2.5% or from about 0.01% to about 2.0 or from about 0.01% to about 1.5% or from about 0.01% to about 1% or from about 0.01% to about 0.5% or from about 0.01% to about 0.1% or from about 0.01% to about 0.05% or from about 0.05% to about 5.0% or from about 0.05% to about 2.5% or from about 0.05% to about 2% or from about 0.05% to about 1.0% or from about 0.05% to about 0.5% or from about 0.1% to about 5.0% or from about 0.1% to about 2.5% for from about 0.1% to about 1.5% or from about 0.1% to about 1.0% or from about 0.1% to 0.5% of the formulation.

Additional compounds may be added to the flavored sprays such as antioxidants which have been found helpful for dogs as well. Vitamin C can improve collagen production for improved gum health. Cracked cell wall chlorophyll can help clean the mouth and reduce odors and Coenzyme Q10 reduces gingival disease in a number of human studies and can benefit dogs as well. Chlorine dioxide or hydrogen peroxide may be used to whiten teeth. Antimicrobials such as honey may also be added to the flavored spray formulation. The amount of compound(s) added should comport with the recommended amount of compound that is safe and effective for a given animal. In some embodiments the of additional compound(s) may be between about 0.01% to about 10% or from about 0.01% to about 7.5% or from about 0.01% to about 5.0% or from about 0.01% to about 2.5% or from about 0.01% to about 2% or from about 0.01% to about 1.0% or from about 0.01% to about 0.1% or from about 0.01% to about 0.05% or from about 0.1% to about 10% or from about 0.1% to about 7.5% or from about 0.1% to about 5.0% or from about 0.1% to about 2.5% or from about 0.1% to about 2% or from about 0.1% to about 1.0% or from about 0.5% to about 7.5% or from about 0.5% to about 5.0% or from about 0.5% to about 2.5% for from about 0.5% to about 1.5% or from about 0.5% to about 1.0%.

Within this disclosure, any indication that a feature is optional is intended provide adequate support (e.g., under 35 U.S.C. 112 or Art. 83 and 84 of EPC) for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” “not including”, “not”, “cannot,” or any combination and/or variation of such language.

Similarly, referents such as “a,” “an,” “said,” or “the,” are intended to support both single and/or plural occurrences unless the context indicates otherwise. For example “a dog” is intended to include support for one dog, no more than one dog, at least one dog, a plurality of dogs, etc. Non-limiting examples of qualifying terms that indicate singularity include “a single”, “one,” “alone”, “only one,” “not more than one”, etc. Non-limiting examples of qualifying terms that indicate (potential or actual) plurality include “at least one,” “one or more,” “more than one,” “two or more,” “a multiplicity,” “a plurality,” “any combination of,” “any permutation of,” “any one or more of,” etc. Claims or descriptions that include “or” between one or more members of a group are considered satisfied if one, more than one, or all of the group members are present in, employed in, or otherwise relevant to a given product or process unless indicated to the contrary or otherwise evident from the context.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While this invention has been particularly shown and described with references to example embodiments thereof, it will be understood by those skilled in the art that the various changes in form and details may be made therein without departing from the scope of the invention encompassed by the appended claims.

Further advantages of the present immunological compositions and adjuvants of the present invention can be achieved by those skilled in the art based upon the embodiments described herein and are thus specifically within the scope of the present invention.

### EXAMPLES

### Example 1

| | | |
|---|---|---|
| Ingredient | mass | Mass percent |

| | | | |
|---|---|---|---|
| Safflower oil | 3600 | mg | 40.8% |
| Cannabidiol (CBD) oil 10% | 2 | mg | 0.028% |
| Hydrogen peroxide (35%) | 15 | mg | 0.170% |
| Flaxseed oil | 4840 | mg | 54.9% |
| Fish oil (33% EPA & 22% | 160 | mg | 1.82% |
| DHA) | | | |
| Rosemary oil | 4.54 | mg | 0.051% |
| Bacon flavor and dessicated | 188.46 | mg | 2.14% |
| liver | | | |

1. A non-aqueous liquid composition for applying to pet food comprising one or more pet food flavorants.

2. The composition of claim 1 wherein the non-aqueous composition comprises more than 80 mass % oil.

3. The composition of claim 1 wherein the non-aqueous composition comprises more than 95 mass % oil.

4. The composition of claim 1 wherein the non-aqueous composition comprises more than 97 mass % oil.

5. The composition of claim 1 wherein the oil is selected from the group consisting of safflower oil, soybean oil, olive oil, flaxseed oil, cottonseed oil, corn oil, canola oil and sunflower oil and combinations thereof.

6. The composition of claim 1 wherein the oil is selected from the group consisting of safflower oil, flaxseed oil and combinations thereof.

7. The composition of claim 6 further comprising cannabidiol oil.

8. The composition of claim 7 further comprising fish oil.

9. The composition of claim 8 further comprising rosemary oil.

10. The composition of claim 9 further comprising hydrogen peroxide.

11. The composition of claim 10 wherein the flavorant is selected from the group consisting of bacon flavor, desiccated liver and combinations thereof.

12. A non-aqueous composition comprising safflower oil, flaxseed oil, cannabidiol oil, fish oil, rosemary oil, hydrogen peroxide, and flavorant.

13. The composition of claim 13 wherein the flavorant is selected from the group consisting of bacon flavor, desiccated liver and combinations thereof.

14. A non-aqueous composition comprising from about 40 to about 60 mass % safflower oil, from about 30 to about 50 mass % flaxseed oil, from about 0.01 to about 0.05 mass % cannabidiol oil, from about 1.0 to about 5 mass % fish oil, from about 0.01 to about 0.1 mass % rosemary oil, from about 0.1 to about 2 mass % hydrogen peroxide, and from about 0.01 to about 5.0 mass % flavorant.

15. The composition of claim 1 further comprising one or more compounds selected from the group consisting of antioxidants, vitamin C, cracked cell wall chlorophyll, coenzyme Q10, antimicrobials, chlorine dioxide and hydrogen peroxide or combinations thereof.

16. The composition of claim 1 wherein the flavorant comprises about 1.0% to about 10% of the composition.

17. The composition of claim 1 wherein the composition is applied to the pet food by spraying.

18. The composition of claim 1 wherein the flavorant comprises about 2.00% to about 5.0% of the composition.

19. The composition of claim 1 wherein the vitamin C comprises about 0.1% to about 1.0% of the composition.

20. The composition of claim 1 wherein the antimicrobial comprises about 0.1% to about 1.0% of the composition.

21. The composition of claim 1 wherein the hydrogen peroxide comprises about 0.1% to about 1.0% of the composition.

22. The composition of claim 1 wherein the chlorine dioxide comprises about 0.1% to about 1.0% of the composition.

23. The composition of claim 1 wherein the cracked cell wall chlorophyll comprises about 0.1% to about 1.0% of the composition.

24. The composition of claim 1 wherein the coenzyme Q10 comprises about 0.1% to about 1.0% of the composition.

25. The composition of claim 1 wherein the antioxidant comprises about 0.1% to about 1.0% of the composition.
